(19) United States
(12) Patent Application Publication
(10) Pub. No.: US 2026/0060938 A1
(43) Pub. Date: Mar. 5, 2026

(54) METHODS OF PROTECTING VISION, FOVEAL ARCHITECTURE, AND EYEBALL SHAPE IN CHILDREN EXPOSED TO NEAR-WORK AND SCREENTIME

(71) Applicant: KEMIN INDUSTRIES, INC., DES MOINES, IA (US)

(72) Inventors: Brenda FONSECA, West Des Moines, IA (US); Joanne LASRADO, Des Moines, IA (US)

(73) Assignee: KEMIN INDUSTRIES, INC., DES MOINES, IA (US)

(21) Appl. No.: 19/315,156

(22) Filed: Aug. 29, 2025

Related U.S. Application Data
(60) Provisional application No. 63/689,434, filed on Aug. 30, 2024.

(51) Int. Cl. A61K 31/047; A61P 27/02

(52) U.S. Cl. CPC A61K 31/047; A61P 27/02

(57) ABSTRACT

The present invention relates to methods of administering ocular nutrients, such as the carotenoids lutein and/or zeaxanthin, to individuals to protect eyeball shape, foveal architecture, vision, and other eye conditions. Another aspect of the present invention relates to the administration of lutein and/or zeaxanthin to individuals, especially during the adolescent years, in order to address the negative effects associated with prolonged periods of near work and/or screentime.

### CROSS REFERENCE TO RELATED APPLICATIONS

The present invention claims the benefit of priority to U.S. Provisional Patent Application No. 63/689,434, filed Aug. 30, 2024, entitled “METHODS OF PROTECTING VISION, FOVEAL ARCHITECTURE, AND EYEBALL SHAPE IN CHILDEN EXPOSED TO NEAR-WORK AND SCREENTIME,” the entire disclosure of which is incorporated herein by reference in its entirety.

### BACKGROUND OF THE INVENTION

Before the COVID-19 pandemic affected digital lifestyles, roughly 19% of children between the ages of 0 and 17 years, were estimated to spend 4 or more hours in front of a digital screen, or approximately 13 million individuals (Child and Adolescent Health Measurement Initiative. 2018-2019 National Survey of Children's Health (NSCH) data query). The prevalence of digital eye strain prior to the pandemic was estimated to be between 22% and 40% (Sheppard & Wolffsohn, 2018). Researchers conducting an open online survey reported that screen time since the pandemic has increased by an average of four hours per day (Bahkir & Grandee, 2020).

Digital eye strain and accommodative fatigue are emerging public health concern, particularly in view of the increased amount of time children are spending in front of digital screens following the COVID-19 pandemic (Bhattacharya et al., 2020). (Lakey-Beitia et al., 2019; Pérez-Gálvez et al., 2018). These issues have become exacerbated as children are spending well over the recommended two hours screen time per day and, excessive exposure to the high energy blue light associated with digital devices has been shown to cause both short term and long term visual damage as well as disruption to the sleep cycle (Jaadane et al., 2015; Knell et al., 2019; Krigel et al., 2016; Maisons-Alfort, 2019; Rideout, 2010; Walsh et al., 2018). A study looking at the effects of screen time via video game playing in over 300 children found that eye strain, including headaches and dizziness was significantly higher in the children reporting higher frequency of video game playing (Rechichi et al., 2017).

Digital eye strain and accommodative fatigue may present in a number of ways, including stress on the foveal architecture and eyeball shape, which in turn may affect vision, visual function, and eye comfort. Improper foveal architecture and eyeball shape can ultimately lead to retinal cell damage, potentially resulting in disorders or conditions, such as macular degeneration, glaucoma, cataracts, retinal detachment, vision loss, and blindness. This is of particular concern for children during the adolescent and teenage years, where it has been observed that the eyes undergo a critical period of rapid growth.

Thus, there remains a need in the art to address, counter, delay, and prevent the negative consequences of increased digital device use and digital eye strain, particularly in children.

### BRIEF DESCRIPTION OF THE INVENTION

The present invention relates to methods of administering ocular nutrients, such as carotenoids (e.g., zeaxanthin, and/or meso-zeaxanthin) to individuals in order to protect their foveal architecture, eyeball shape, vision, and other eye conditions. Another aspect of the present invention relates to methods to address, counter, delay, or prevent the negative effects of prolonged near work and screentime in individuals, especially teenagers.

### BRIEF DESCRIPTION OF THE DRAWINGS

FIG. 1 provides an overview of the study outline.

FIGS. 2A-2E describe the baseline characteristics of participants for (A) age, (B) screen time, (C) digital eye strain, (D) macular pigment-HFP, and (E) macular pigment-HB. N=59.

FIG. 3 represents the final data set used for the mixed model repeated measures statistical analyses (CONSORT Diagram).

FIGS. 4A-4B shows the mean change in macular pigment values over time assessed via (A) heterochromatic flicker photometry and (B) Haidinger's Brush degree of polarization. Error bars indicate ±1 standard error of the mean. N=54. Asterisk (*) indicates statistically significant difference between groups at p<0.05.

FIG. 5 shows the mean change in digital eye strain values over time assessed via Visual Fatigue Scale. Error bars indicate ±1 standard error of the mean. N=54. Asterisk (*) indicates statistically significant difference between groups at p<0.05.

FIGS. 6A-6F shows the mean change in digital eye strain values over time assessed via Visual Fatigue Scale: (A) problems seeing, (B) strange feeling, (C) tired eyes, (D) feeling numb, (E) headache, (F) feeling dizzy. Error bars indicate ±1 standard error of the mean. N=54. Caret (\hat{ }) indicates a significant trend between groups at p<0.10 from post-hoc independent samples t-test of change from baseline to day 180.

### DETAILED DESCRIPTION OF THE INVENTION

Globally, the adolescent population appears to be at high-risk for near work related eye health conditions. As used herein “near work” describes any activity that includes focusing on an object within an arm's length from the eyes (e.g., use of a smart phone or electronic device, reading a book). For instance, research have identified that near work increases the risk of myopia. As described in greater detail herein, the adolescent population presents a unique opportunity for potential intervention. Of all of the age groups, adolescents spend the greatest amount of time on digital devices, with recent studies showing teenagers in the United States spending over 8 hours per day on recreational digital devices such as phones, computers, and gaming consoles. Additionally, adolescence is a time of rapid neuronal development and plasticity, meaning that the neural tissues of the eye and brain are potentially susceptible to small environmental changes and that these environmental influences during this age can have a life-long impact regarding an individual's health trajectory.

Macular carotenoids from the diet represent one approach to protecting the retina from long term damage caused by excessive near work. One important and lesser known macular carotenoid that comes from fruits and vegetables is lutein (Bernstein et al., 2016). Lutein is an orange-colored pigment found in many of the brightly colored fruits and vegetables in the human diet (Eisenhauer et al., 2017). The human body cannot produce lutein on its own and must obtain lutein from either the diet or supplementation (Bernstein et al., 2016). Some of the fruits and vegetables with the highest concentrations of lutein include dark green leafy vegetables such as kale, spinach, broccoli and collard greens, as well as beans, butternut squash and oranges (Humphries & Khachik, 2003). When lutein is consumed from the diet or supplementation, the colored molecule gets deposited in the eye, skin and brain where it has been found to play a key role as an antioxidant, anti-inflammatory, and blue light absorber and/or filter (Bernstein & Ranganathan, 2021).

Research has shown that a daily intake of at least 10 mg of lutein from the diet is beneficial for both the eyes and the brain (Age-Related Eye Disease Study 2 Research et al., 2014; Hammond et al., 2014; Jia et al., 2017; Mares, 2016). Unfortunately, due to the lack of clinical trials with children, there is currently no recommended daily intake values for individuals under the age of 18 years. However, studies indicate that in the United States, the average daily intake of lutein is around only 1 mg per day (Johnson et al., 2010). The situation is not much better globally, with average intakes in the European Union ranging from 0.5 mg to 4 mg, for example (Granado et al., 2007).

In a survey of almost 200,000 individuals 15 years of age and older, only 18% of the sample met the World Health Organization's recommended daily intake of at least five servings of fruit and vegetables which contain lutein (Frank et al., 2019). Accordingly, there is an unresolved need to address the fact that children are not getting enough lutein in their diets which can lead to age related eye and brain impairment (Age-Related Eye Disease Study 2 Research et al., 2014; Christensen et al., 2018; Eisenhauer et al., 2017; Amy C. Long et al., 2019). The recommended daily intake of 10 mg of the nutrient lutein (required for eye and brain function) is not being met by most children (Kim et al., 2014; Thurnham, 2007). In the United States alone, 99% of children between the ages of 2 to 18 years are not eating the recommended daily amount of dark, green leafy vegetables. (Krebs-Smith et al., 2010). Even more alarming, over 90% of children in the United States do not eat the recommended daily amount of vegetables (Kim et al., 2014). The intake does not appear to be better from a global perspective with a recent national survey conducted in England found that only eight percent of children between the ages of 11 and 18 consumed the 5-a-day recommended daily intake of fruits and vegetables (Bates, 2016). In fact, a study of 28 low and middle income countries found that the over eighty percent of individuals 15 years and older failed to meet the daily recommended intake levels (Frank et al., 2019).

There are approximately 21.4 million children between the ages of 10 and 14 years currently living in the United States (ACS Demographic and housing estimates, 2019). This age group represents the U.S. demographic with the lowest healthy eating index (HEI) of all age groups with an average 52 out of a possible 100 along with a reported approximate vegetable intake of only ½ cup per week of dark green leafy vegetables, known for their high content of lutein and zeaxanthin (Abdel-Aal el et al., 2013; Dietary Guidelines for Americans, 2020-2025., December 2020.; Eisenhauer et al., 2017).

Against this backdrop of increased exposure to digital devices and eye strain and a diet with the lowest healthy eating index, the inventors have further determined that in this adolescent population, lutein and/or zeaxanthin may be even more important in the developing eye and brain than in the adult body. Existing data suggest preferential uptake of lutein in juvenile tissue and developing organisms. Furthermore, there is compelling evidence that increased levels of lutein and/or zeaxanthin at an early age can result in positive long-term benefits. For example, multiple cross-sectional studies conducted in children have found that higher levels of lutein and/or zeaxanthin in the eye, measured as macular pigment optical density (MPOD) was strongly correlated with measures of intelligence, academic achievement and cognitive control (Hassevoort et al., 2017; Saint et al., 2018; Walk et al., 2017). In addition, a series of studies conducted in primates demonstrated that a diet deficient in lutein led to fundamental deficits in eye development and visual function while restoring lutein to the diet was able to correct many of the problems (Leung et al., 2004; Neuringer et al., 2004). High levels of lutein in the retina are also predicted to lead to positive health outcomes later in life, and in fact, studies have shown that MPOD levels tend to decrease with age, making it all the more critical to ensure high levels of lutein from as early age as possible (Arunkumar et al., 2018; Ji et al., 2015).

Even more alarming, studies have shown a strong correlation between low intake of lutein and age-related macular degeneration (Wu et al., 2015), as well as with cognitive decline and Alzheimer's disease later in life. With a globally aging population, if this deficit is not addressed adequately early on in life, there will be substantial public health consequences. One study predicted that if individuals were to consume the recommended levels of lutein daily, there would be a seven percent reduced risk for age related eye disease and a potential savings of over five billion US dollars annually due to costs of required medical care (Frost & Sullivan, October 2017).

Despite these alarming trends, there are limited interventional studies in children to offer guidance on lutein and/or zeaxanthin intake, either through diet or supplementation. To the inventors' knowledge, there are no published studies to date investigating the impact of lutein and/or zeaxanthin supplementation on vision and eye health in healthy adolescents or confirmation that lutein and/or zeaxanthin intake will result in increased macular pigment levels in the eye. The inventors have for the first-time identified and described previously unknown benefits of lutein supplementation in children, namely, protecting vision, reducing abnormal eyeball shape changes, and preserving foveal architecture of children exposed to digital screens and near work. And, namely, the benefits of lutein and/or zeaxanthin supplementation for supporting foveal architecture and integrity to preserve ocular shape and structure, which reduces biomechanical stress and perception of eye strain, accommodative fatigue and ultimately vision.

These conditions include but are not limited to damage to the retinal nerve layer, improper eyeball elongation, eyeball shape change, impaired vision, eye strain, accommodative fatigue, screentime fatigue, biomechanical stress, dry eye, strabismus, and/or irregular anatomical development or damage of the fovea or foveal architecture are all problematic issues that are exacerbated by exposure to digital screens and near work. The methods disclosed herein help prevent, reduce, delay, and mitigate these conditions.

One proposed benefit of lutein and/or zeaxanthin accumulation within the retina is the protection from short-wavelength high energy blue light present in both sunlight and digital device screens. Due to the high energy nature of blue light, excessive exposure can lead to oxidative stress in the retina and damage to photoreceptor cells. A study in primates showed that when the animal was deficient in lutein, blue light exposure significantly damaged the retina and, subsequently, the macula. (Barker et al., 2011).

One aspect of the present invention relates to administering carotenoids, such as lutein and/or zeaxanthin, to an individual in order to protect and maintain the integrity of an individual's eyes, including the eye structure, such as preserving eyeball shape and/or preventing elongation. Administration of carotenoids, such as lutein and/or zeaxanthin, also benefits the surround ocular tissues that may affect vision such as the uveal tissues, sclera, and the foveal architecture.

In certain embodiments, the administration of one or more ocular nutrients, such as lutein and/or zeaxanthin, protects the eye by mechanisms of reduced oxidative stress, reduced inflammation, cellular signaling, neurotrophin support, and gene regulation.

In certain embodiments, eyeball elongation is prevented by the accumulation of carotenoids in the uveal tissues, which include the choroid, the iris and the ciliary bodies. The choroid sits between the retina and sclera. A thickened choroid is correlated with a reduced risk and reduced progression of eyeball shape lengthening. Thus, by increasing the level of carotenoids, the choroid is strengthened and protected.

The foveal architecture refers to the specialized structure of fovea, which is the central part of the retina. The fovea is responsible for visual acuity-allowing for fine details and color to be distinguished. The fovea is devoid of rod photoreceptors, but contains cones which are directly stimulated to achieve this visual acuity. Carotenoid pigments, such as lutein and/or zeaxanthin may accumulate in the fovea. Irregular anatomical development of the fovea is detrimental to eye health and vision, and may cause irregular eyeball shape.

Proper regulation of dopamine also plays a role in controlling eye growth and maintaining correct eyeball shape. Extended periods of near work, such as using digital devices and screen time are associated with dopamine deficiency in the eye. This disclosure contemplates that carotenoids can help preserve and regulate dopamine in the eye and help prevent a dopamine deficiency in the eye.

As described above, the eyes of adolescents, or teens and pre-teens, undergo significant development and growth during this critical stage of life. The inventors have surprisingly discovered for the first time that this developmental phase appears to affect the accumulation of lutein and/or zeaxanthin. Specifically, the accumulation of lutein and/or zeaxanthin happens rapidly-more quickly than seen in other age groups and populations. The accumulation of lutein may also be more rapid due to a compromised or stretched retina resulting from the high amount of digital device use or near work. Thus, both the developmental time frame and a compromised retina may “prime” the retina to receive lutein and/or zeaxanthin. Additionally, a lower dose of lutein and/or zeaxanthin may be required when compared with other age groups and populations. This disclosure further contemplates that lutein and/or zeaxanthin supplementation can be used as a preventative measure to intervene before damage occurs in children.

Ocular nutrients may be used for the methods described herein. Such ocular nutrients may include antioxidant vitamins, carotenoids, antioxidant minerals, natural antioxidant extracts, and fatty acids alone or in combination. Such antioxidant vitamins may include vitamins A, C, and/or E. Such carotenoids may include lutein, zeaxanthin, beta cryptoxanthin, beta carotene, astaxanthin, retinoids, retinal, retinaldehyde, capsanthin, astaxanthin, and/or meso-zeaxanthin, and esters thereof. Such antioxidant minerals may include zinc, copper, and/or selenium. Such natural antioxidant extract may include polyphenols, quercetagetin, quercetin, anthocyanidins, and/or turmeric (containing rosmarinic acid). Such fatty acids may include omega 3 fatty acids and/or short chain fatty acids.

One aspect of the present invention relates to the supplementation of ocular nutrients to a population in need thereof, which includes but is not limited all children, and according to at least one embodiment, the population includes one or more subjects experiencing adolescence, such as teenagers (i.e., 13 to 18 years of age), pre-teenagers (i.e., 8 to 12 years of age), and younger children (i.e., 7 years of age or younger).

In certain embodiments, the supplementation should be added in a therapeutically effective amount in a subject. In an embodiment, a therapeutically effective amount of the nutrient, antioxidant, or vitamin is between about 0.02 (1 IU) and 15 mg (150 IU) per kilogram of body weight of the subject per day. In an embodiment, a therapeutically effective amount of the nutrient, such as lutein and/or zeaxanthin, is between about 0.0001 and 2 mg per kilogram of body weight of the subject per day. In an embodiment, a therapeutically effective amount of nutrient, such as lutein and/or zeaxanthin, is in the range of between about 0.0001 and 5 mg per kilogram of body weight of the subject per day.

In certain embodiments, an effective amount of ocular nutrient supplementation is in the range of about 0.1 mg/day to about 30 mg/day, for instance between about 0.5 to about 15 mg/day, 0.5 to about 10 mg/day, or about 5 to 10 mg/day, such as about 0.5 mg/day, 0.6 mg/day, 0.7 mg/day, 0.8 mg/day, 0.9 mg/day, 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 20 mg/day, 25 mg/day, or 30 mg/day.

In certain embodiments the ocular nutrient supplementation dose provided for the disclosed methods is at least 0.5 mg/day, 1 mg/day, at least 2 mg/day, at least 3 mg/day, at least 5 mg/day, at least 6 mg/day, at least 7 mg/day, at least 8 mg/day, at least 9 mg/day, at least 10 mg/day, at least 11 mg/day, at least 12 mg/day, at least 13 mg/day, at least 14 mg/day, at least 15 mg/day, at least 20 mg/day, at least 25 mg/day, or at least 30 mg/day.

According to one aspect of the invention, the subjects receiving supplementation under the methods described herein may be children. In certain embodiments, the population includes individuals during adolescence (typically, spanning 9 to 19 years of age) and young adulthood (typically, up to 24 years of age), and includes but is not limited to teenagers (i.e., 13 to 18 years of age), pre-teenagers (i.e., 8 to 12 years of age), and in some instances younger than 8 years of age. It should be appreciated that young adults and adults may also benefit from the methods described herein.

In certain embodiments, the individuals or subjects with high usage of digital devices (i.e., greater than 4 hours average per day) may particularly benefit from the methods described herein.

In certain embodiments, the method comprises carotenoid supplementation or administration to an individual or subject provides protection through anti-oxidant properties, anti-inflammatory properties, blue light filtering, and an increase in dopamine levels in an individual or subject, which may slow shape change within an eyeball.

In certain embodiments, the administration of one or more ocular nutrients, such as lutein and/or zeaxanthin, to an individual or subject prevents or delays damage associated with digital screens, near work, and blue light. In an embodiment, the administration of one or more ocular nutrients to an individual or subject, such as lutein and/or zeaxanthin, reverses damage to the eye from exposure to digital screens, near work, and blue light.

In certain embodiments, the administration of one or more ocular nutrients, such as lutein and/or zeaxanthin, to an individual or subject protects the vision of an individual or subject with exposure to digital screens and near work, including under conditions causing damage to the retinal nerve layer, improper eyeball elongation, improper eyeball shape, biomechanical stress, damage to the foveal architecture, and/or irregular anatomical development of the fovea.

In certain embodiments, the administration of one or more ocular nutrients, such as lutein and/or zeaxanthin, to an individual or subject reduces abnormal eyeball shape changes of an individual or subject with exposure to digital screens and near work, including under conditions causing damage to the retinal nerve layer, and/or irregular anatomical development of the fovea.

In certain embodiments, the administration of one or more ocular nutrients, such as lutein and/or zeaxanthin, to an individual or subject prevents or delays abnormal eyeball shape changes of an individual or subject person with exposure to digital screens and near work, including under conditions causing damage to the retinal nerve layer, improper eyeball elongation, improper eyeball shape, biomechanical stress, damage to the foveal architecture, and/or irregular anatomical development of the fovea.

In certain embodiments, the administration of one or more ocular nutrients, such as lutein and/or zeaxanthin, to an individual or subject preserves the foveal architecture of an individual or subject with exposure to digital screens and near work, including under conditions causing damage to the retinal nerve layer, improper eyeball elongation, and/or irregular anatomical development of the fovea. In an embodiment, the preservation occurs prior to damage of the foveal architecture. In certain embodiments, the preservation occurs prior to irreversible damage of the foveal architecture.

In certain embodiments, the administration of one or more ocular nutrients, such as lutein and/or zeaxanthin, to an individual or subject minimizes or mitigates damage to the foveal architecture of a person with exposure to digital screens and near work, including under conditions causing damage to the retinal nerve layer, improper eyeball elongation, and/or irregular anatomical development of the fovea.

In certain embodiments, the administration of one or more ocular nutrients, such as lutein and/or zeaxanthin, to an individual or subject prevents, delays, minimizes, or mitigates eye strain and/or screentime fatigue of a person with exposure to digital screens and near work, including under conditions causing damage to the retinal nerve layer, improper eyeball elongation, and/or irregular anatomical development of the fovea.

Another aspect of the present invention relates to healthy lifestyle interventions, i.e., supplementation or administration of carotenoids, such as lutein and/or zeaxanthin, to ensure adequate macular pigment levels in a vulnerable population, such as children during the adolescence phase, wherein the supplementation or administration of carotenoids is in an amount effective to provide protection against potential damage due to exposure to digital screens, blue light, and near work that can lead to eye conditions including vision (myopia or hyperopia), eye strain, biomechanical stress, dry eye, strabismus, damage to foveal architecture, irregular development of fovea, eyeball shape change, improper eyeball elongation, screentime fatigue, and retinal nerve cell layer damage.

In certain embodiments, the methods described herein include subjects with one or more conditions. For instance, where the subject is experiencing one eye condition, but not a second eye condition, the administration of a carotenoid may prevent or delay the second eye condition from developing or progressing. Simultaneously, the first eye condition may be reduced or mitigated from the same administration.

In certain embodiments, the administration of the ocular nutrient, such as lutein and/or zeaxanthin, may be distributed in any suitable medium or form, including, but not limited to a gummy, tablet, capsules, soft gel, chewing gum, yogurt, or carbonated beverage.

In certain embodiments, the carotenoid, such as lutein and/or zeaxanthin, may be sourced from a natural source, including but not limited to the group consisting of marigold, paprika, Chinese lantern, spinach, kale, collard greens, yellow sweet corn, peas, brussel sprouts, broccoli rabe, asparagus, arugula, avocado, eggs, pistachios, winter squash, pumpkin, sweet potatoes, carrots, or goji berries.

In certain embodiments, the carotenoid, such as lutein and/or zeaxanthin, may be administered with other important nutrients and vitamins, such as omega 3 fatty acids, which may provide a synergistic effect on eye and brain benefits.

### Example 1: The “LuTEEN” Trial: Effects of Daily Lutein Supplementation on Macular Pigment and Digital Eye Strain in Healthy Youth (Pre-Teen and Teen)—A Randomized, Placebo-Controlled Study

The objective of this study was to determine the effects of daily supplementation with 5 mg lutein or placebo on the macular pigment density of healthy participants between the ages of 8 and 16 years who were exposed to four hours or more of daily digital screens. In addition, the study was designed to track changes in digital eye strain over the course as well as baseline values of health literacy and vegetable self-efficacy.

The population of teens and pre-teens was identified to investigate the impact of supplemental lutein on macular pigment density in the retina and related effects on eye and brain health, where the age group has a reported low level of dietary lutein (Dietary Guidelines for Americans, 2020-2025., December 2020) intake along with excessive screen time exposure. In addition, this age group can easily perform the non-invasive tests designed to measure lutein levels in the eye (Temple et al. 2015; Wooten et al., 1999) and to the inventors' knowledge would be the first teenage youth population to be studied via an interventional study with supplemental lutein.

Importantly, macular pigment composed of both lutein and zeaxanthin can be measured via non-invasive methods to indicate the amount of lutein and zeaxanthin deposited in the eye and correlates with lutein levels in the brain (Bernstein et al., 2016; Vishwanathan et al., 2016). Heterochromatic flicker photometry (HFP) is the most commonly used psychophysical technique used to measure macular pigment optical density (MPOD) clinically and works by comparing flicker perception of two lights at different wavelengths. The participant response provides an estimate of macular pigment optical density (MPOD) which is an indication of macular pigment levels in the fovea center of one eye. HFP measures macular pigment at a single point in the retina and can be challenging for younger participants to perform with typical standard deviations of 0.2 to 0.3 optical density units (McCorkle et al. 2015). Commercially available devices such as the MPSII QuantifEye Macular Pigment Device can be used to implement the HFP technique. A newer psychophysical measurement tool is the MPEye. The MPEye is a device that uses the entoptic phenomenon of Haidinger's brushes (HB) to measure lutein and zeaxanthin concentrations in the eye (Temple et al., 2015). The participant looks through a view finder with both eyes and indicates the direction of a spinning yellow bowtie shape. The ability to see the image is determined by macular pigment concentration along the henle fibers and provides a measure of macular pigment density across the entire macula. While both methods are validated, HFP is reliant on a participants' ability to perceive flickering lights in a single eye while MPEye measures macular pigment density across the entire retina in both eyes and relies on the participants ability to observe a spinning yellow light. In addition, the MPEye detects macular pigment levels across the entire retina whereas HFP assesses macular pigment at a single point in the retina. The distribution of macular pigment following lutein supplementation in healthy pre-teen and teen youth has not yet been studied and therefore it is not known if HFP or MPEye is the more sensitive measure to detect changes in macular carotenoids over time. Therefore, depending on the age of the child one method may be easier to administer and may capture developmental changes more completely. Nonetheless, both MPOD measurement methods are painless, can be completed in approximately 5-10 minutes of time and have been shown to be valid and reliable measures of macular pigment in school-aged children (Cannavale et al., 2019; Hassevoort et al., 2017; Saint et al., 2018; Walk et al., 2017).

### Methods

### Study Design

This was a six-month, prospective, randomized, double-blind, placebo-controlled parallel trial in healthy children and adolescents with daily digital screen exposure conducted at a single site (Des Moines, IA). The study consisted of three visits to the study site (baseline, 90 and 180-day) along with bi-weekly text check-ins. All visits conformed to standards of the use of human participants in research as outlined in the Helsinki Declaration and all study procedures were reviewed and approved by an external Institutional Review Board (Salus IRB No. IRB00013544). The study was prospectively registered at ClinicalTrials.gov (NCT05314647).

### Study Participants

Fifty-nine participants between 8 and 16 years of age were recruited for this study. The inclusion criteria included being between the ages of 8 and 16 years on the day of enrollment, with self-reported exposure to digital screens of four hours or more per day on average, and were in good general health and with no lutein or zeaxanthin supplement use during the prior six months. The four hours or more digital screen inclusion criteria was selected based on research showing that screen time greater than 4-6 hours daily may degrade eye function significantly (Yang et al., 2020). In addition, a recent study in children with a mean age of 13 years revealed that the average screen time per day was 3.9 hours, a major jump from the pre-COVID pandemic average of 1.9 hours (Mohan et al., 2021). Furthermore, researchers have found that children with greater than 5 hours of screen time per day were at a significantly increased risk for digital eye strain (Mohan et al., 2021).

### Sample Size

The study was designed to have a power of 80% (β=0.20) assuming a moderate effect size of 0.6 with lutein supplementation compared to placebo. The power analyses were based on related studies (Hassevoort et al., 2017; Renzi-Hammond et al., 2017; Saint et al., 2018; Stringham et al., 2017; Walk et al., 2017). Specifically, data from Stringham et al., 2017 reporting significant improvements in MPOD values in healthy young adults with four hours of screen time or more per day after six months of supplementation assessed via heterochromatic flicker photometry (0.383 and 0.472 for placebo and treatment MPOD values respectively and 0.132 for placebo standard deviation) was used to inform the analyses. The power analyses suggested a total sample size of 70 (35 per group) to account for possible attrition was required to reach power of 80% (β=0.20) with a two-sided alpha of 0.05.

### Study Intervention

The participants were randomized to receive either 5 mg lutein (FloraGLO® Lutein, Kemin Industries, Des Moines, IA, LOT No. UT20100010) or an identical placebo without the active in a sugar-free gummy format. The pectin gummies were manufactured in a GMP facility by TopGum Industries, LTD, Sderot, Israel No lutein or zeaxanthin was detected in the placebo gummies at any time point, as expected. The lutein gummies were found to contain, on average, 6.89 mg lutein and 0.53 mg zeaxanthin per gummy at time zero, which slightly exceeds the target of 5 mg lutein per gummy most likely due to overage added during production. The lutein gummies contained a ratio of 13:1 lutein to zeaxanthin, which is consistent with the ratio found in FloraGLOR Lutein material (Kemin Industries, Des Moines, IA). Gummies were analyzed at both 5 months and 8 months after time zero (equivalent to 7 months and 10 months after gummy manufacture date). Lutein was found to be 102% and 101% of time zero content at the measured time points, respectively. Zeaxanthin was measured at 104% and 102% of time zero content at the measured time points, respectively. These data confirm that the active components in the gummies used for the clinical trial have remained stable throughout the study.

### Randomization and Study Procedures

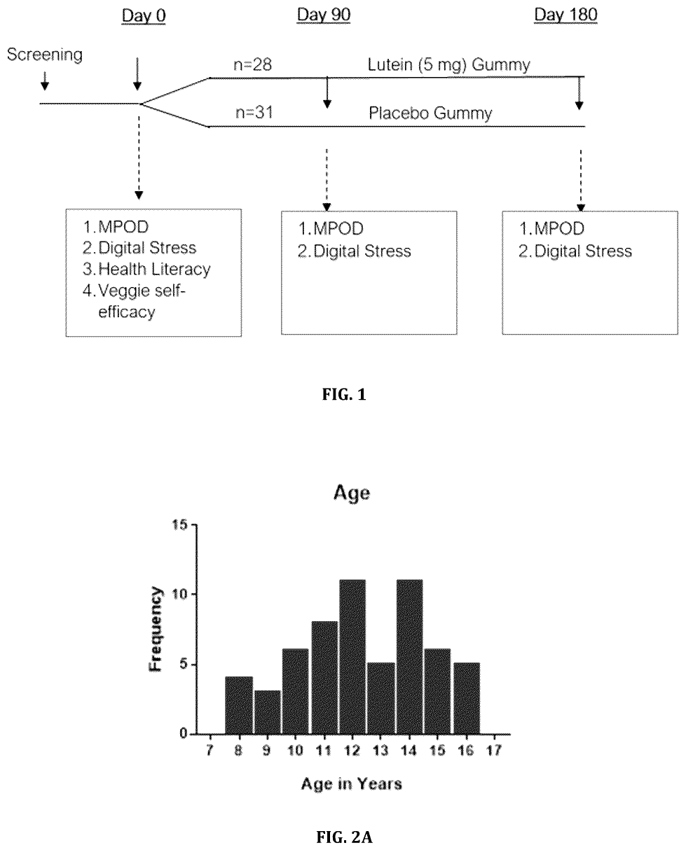

The study consisted of three visits to the study site (baseline, 90 and 180-day) along with bi-weekly text check-ins. FIG. 1 provides an overview of the study outline. Screening phone calls were made to potential participants to determine eligibility for inclusion in the study. The phones calls were conducted with the parent or guardian of the children and included questions on age, digital device use, lutein/zeaxanthin supplement use and overall health. If eligibility criteria were met, the parent and participant were invited to come to the study site for baseline assessment and enrollment in the study. At the baseline visit, both parents and participants completed the health literacy assessment. Participants took the macular pigment measurements and digital eye strain survey.

After completing the baseline screening, each participant was assigned to a group based on a simple randomization procedure using a computer generated random number sequence (Kim & Shin, 2014). Randomization was based on a variable block randomization algorithm via Castor EDC Software (Castor v2024.2.0.1, Castor Research Inc, Amsterdam, The Netherlands). Block sizes were set to 4, 6, and 8 and participants were stratified based on age group (8-10, 11-13 and 14-16 year olds) and sex (female, male). Based on randomization, participants were provided with an opaque Bottle A or Bottle B of gummies with a total of 100 gummies to last for the next 90 days. They were instructed to take one gummy per day with food. Bi-weekly text messages were sent to the participant's parents to check on compliance and any potential adverse events to the study product. Participants returned to the lab on days 90 and 180 and the same measurements were made with the participants. Product bottles were collected, and the remaining gummies were counted to determine compliance. No additional data were collected from parents at either of the two subsequent time points.

Compliance percentage was defined as the number of gummies remaining in the bottle divided by the number of gummies when the bottle was dispersed and then multiplied by 100 to give a percentage. Compliance was assessed by counting the number of gummies returned in the product bottles on days 90 and 180 and was encouraged via weekly text messages to the participants. Compliance was defined as between 80% and 120% of gummies consumed at the two assessment time points.

### Outcome Measures

### Primary Outcome

The primary outcome of the clinical trial was the change in macular pigment density observed at 90 and 180 day compared to baseline using two different validated instruments; the QuanitfEye based on heterochromatic flicker photometry (HFP) (EyePromise, Chesterfield MO, USA) and the MPEye based on the concept of Haidinger's brushes (HB) (Azul Optics, Bristol UK). Both methodologies utilize a psychometric approach to determine the density of the macular pigment and allow for repeated non-invasive assessments of the lutein and zeaxanthin content of the retina (Wooten et al., 1999: Temple et al., 2015). Both procedures have been carried out in children and adults (Chew et al., 2004; Saint et al., 2018; Walk et al., 2017: Sangani et al. 2024) and have been shown to be safe, comfortable, and painless.

### Secondary Outcomes

The secondary outcomes assessed included health literacy, veggie efficacy and digital eye strain. Health literacy was assessed in both the children and parents using the Newest Vital Sign (NVS) assessment tool (Driessnack et al., 2014; Weiss et al., 2005). The assessment, which takes approximately three minutes, requires the participant to look over a nutrition facts panel for ice cream and answer six questions about the information provided in the facts panel. The instrument has been found to have high internal consistency and to correlate well with other measures of health literacy (Weiss et al., 2005). The instrument has been studied in children and found to perform well (Chari et al., 2014; Driessnack et al., 2014; Guo et al., 2018; Okan et al., 2018; Warsh et al., 2014). Overall, the NVS has demonstrated sensitivity as an assessment of health literacy in parents and found to correlate well with real life health outcomes such as emergency room visits and adolescent obesity (Chari et al., 2014; Morrison et al., 2014). Veggie self-efficacy was assessed via a 20-point questionnaire asking about the participants habits related to vegetable consumption. The survey takes approximately three minutes to complete with a minimum possible score of 20 and maximum of 120. The instrument has been validated in populations as young as 8 years of age (Sharma et al. 2014).

The instrument for digital eye strain consisted of a six question validated assessment measured on a frequency scale, similar to the visual fatigue scale used in adults (Benedetto et al., 2013; Saoji et al. 2024; Cavusoglu et al, 2023; Swathi et al. 2022). The six items assessed on a 4-point Likert scale consisted of the following questions: 1) I have difficulties in seeing; 2) I have a strange feeling around the eyes; 3) My eyes feel tired; 4) I feel numb; 5) I have a headache; 6) I feel dizzy looking at the screen (Benedetto et al., 2013; Stringham et al., 2017). These questions representa sub-set of symptoms assessed in the Computer Vision Syndrome Questionnaire, a validated instrument for use in measuring digital eye strain in adults (Cantó-Sancho et al., 2020; Seguí Mdel et al., 2015; Sheppard & Wolffsohn, 2018) because research shows that children and adolescents are unable to correctly report on all the symptoms identified in the Computer Vision Syndrome Questionnaire (Hu et al., 2013; De-Hita-Cantalejo et al., 2020; Ichhpujani et al., 2019).

### Statistical Analysis

Data were analyzed using SPSS statistical software package (IBM Corp. Version 29.0.1.0 (171)) with p<0.05 set as the criterion for significance. Only participants who met the compliance criteria were included in the analysis. A mixed model repeated measures (MMRM) was conducted on the per protocol population of 54 participants with the removal of 5 participants from the intent to treat population due to a gummy compliance level less than 80%. For each subject, change from baseline was calculated for the 90 day and 180-day visits with treatment, visit, and treatment by visit interaction treated as fixed effects, participant as a random effect and baseline macular pigment values as a covariate. A variance components covariance structure was used with a maximum likelihood estimation and Satterthwaite approximation for degrees of freedom. Pairwise comparisons on the estimated marginal means were conducted using Bonferroni tests to account for multiple comparisons. Identical analyses were conducted on digital eye strain scores using baseline VFS score as a covariate. Descriptive statistics were used to assess subject characteristics at baseline with all participants included (n=59) and independent samples t-tests or Pearson chi-square tests were used to determine if any group differences were present at baseline.

### Results

### Participant Characteristics and Compliance

All 59 participants screened for the study between the ages of 8 and 16 years were deemed eligible to participate and were recruited for the study. The average age of the participants was 12.3 years (SD=2.23) with 59% of the participants in the pre-teen/youth category (8-12 years) and 41% in the adolescent age range (13-16 years). Participants were evenly split between female (n=30) and male (n=29) with White or Caucasian making up the primary racial/ethnic category (n=48). Table 1 provides detailed demographics of the participants at baseline.

TABLE 1
Participant Demographics at baseline (n = 59)

| | | | |
|---|---|---|---|
| Overall Total | Placebo | Lutein | p-value |

Age in years

| | | | | | | | |
|---|---|---|---|---|---|---|---|
| Mean (±SD) y | 12.3 | (±2.23) | 12.25 | (±2.31) | 12.43 | (±2.18) | 0.755¹ |

| | | | | |
|---|---|---|---|---|
| 8-10 y | 25.4% | (n = 15) | n = 8 | n = 7 |
| 11-13 y | 45.8% | (n = 27) | n = 14 | n = 13 |
| 14-16 y | 28.8% | (n = 17) | n = 9 | n = 8 |

Gender

| | | | | | | | |
|---|---|---|---|---|---|---|---|
| Female | 50.8% | (n = 30) | 53.3% | (n = 16) | 46.7% | (n = 14) | 0.902² |
| Male | 49.2% | (n − 29) | 51.7% | (n = 15) | 48.2% | (n = 14) | |

Hours Screen time (h)

| | | | | | | | |
|---|---|---|---|---|---|---|---|
| Mean (±SD) h | 5.51 h | (±1.5) | 5.61 h | (±1.7) | 5.44 h | (±1.3) | 0.587¹ |

| | | | |
|---|---|---|---|
| Min | 4 h | 4 h | 4 h |
| Max | 10 h | 10 h | 8 h |

Racial/Ethnic group

| | | | | | |
|---|---|---|---|---|---|
| White | 81.4% | (n = 48) | n = 23 | n = 25 | 0.358² |
| Asian | 11.9% | (n = 7) | n = 5 | n = 2 | |
| Hispanic or Latino | 3.4% | (n = 2) | n = 1 | n = 1 | |
| Mixed | 3.4% | (n = 2) | n = 2 | n = 0 | |
| Black or African American | 0% | (n = 0) | n = 0 | n = 0 | |
| American Indian or Alaska | 0% | (n = 0) | n = 0 | n = 0 | |
| Native | | | | | |
| Native Hawaiian/Other | 0% | (n = 0) | n = 0 | n = 0 | |
| Pacific Islander | | | | | |

Baseline Macular Pigment Values

| | | | | |
|---|---|---|---|---|
| Heterochromatic Flicker | 0.547 ± 0.144 | 0.555 ± 0.160 | 0.539 ± 0.129 | 0.684¹ |
| Photometry: Mean (±SD) | (n = 58) | (n = 30) | (n = 28) | |
| Haidinger's Brush degree | 5.136 ± 2.4563 | 5.32 ± 2.257 | 4.93 ± 2.721 | 0.546¹ |
| of polarization: Mean | (n = 59) | (n = 31) | (n = 28) | |
| (±SD) | | | | |

Eye Strain

| | | | | |
|---|---|---|---|---|
| Digital Eye Strain (VFS) | 10.88 ± 3.09 | 10.81 ± 2.70 | 10.61 ± 2.74 | 0.847 |
| | (n = 59) | (n = 31) | (n = 31) | |

Health Knowledge

| | | | | |
|---|---|---|---|---|
| Parent Health Literacy | 5.46 ± 0.87 | 5.48 ± 0.98 | 5.45 ± 0.76 | 0.925¹ |
| (NVS) | (n = 41) | (n = 21) | (n = 20) | |
| Child Health Literacy | 3.65 ± 2.16 | 3.71 ± 2.23 | 3.58 ± 2.12 | 0.818¹ |
| (NVS) | (n = 54) | (n = 28) | (n = 20) | |

¹Two-sided p-values from independent samples t-test with equal variances assumed
²Pearson Chi-square test with two-sided p-value

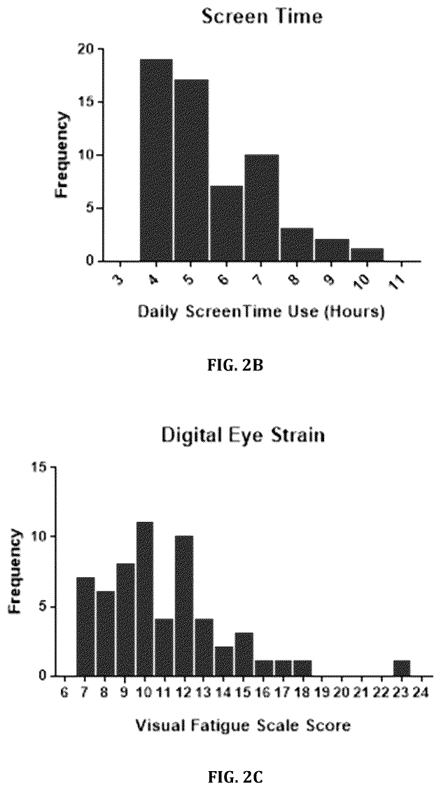

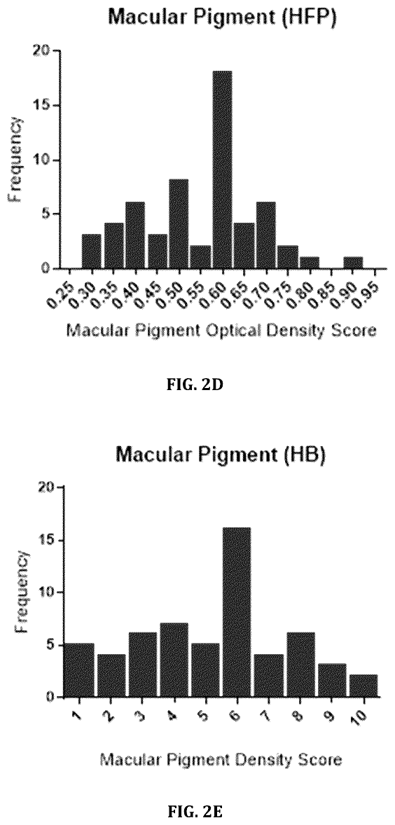

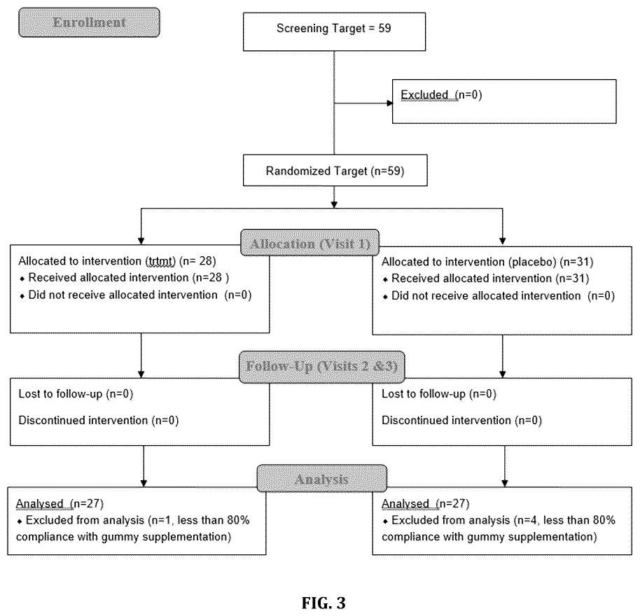

Independent sample t-tests were conducted to determine any baseline differences in demographic data between the treatment and placebo groups and all tests resulted in non-significant probabilities (p>0.35 for all). Mean self-reported screen time for all participants at baseline was 5.4 hours (SD=1.3) and mean digital eye strain was 10.9 (SD=3.1). The frequency distributions for age, screen time, digital eye strain and baseline macular pigment values can be seen in FIGS. 2A-2E.

Based on the gummy count, the participants were 94.56% compliant with a standard deviation of 10.3%. However, five participants did not reach the threshold of inclusion by maintaining a compliance greater than 80% and less than 120%. All five participants had less than 80% compliance (49.7%, 76.41%, 77.49%, 77.84% and 78.92%). As such, these participants were removed from the per protocol analysis, bringing the average compliance in the per protocol population to 96.65% with a standard deviation of 7.3%. Of the five participants removed from further analysis, four were in the placebo group and one was in the treatment group. The CONSORT diagram (FIG. 3) represents the final data set used for the mixed model repeated measures statistical analyses.

### Health Literacy and Veggie Self-Efficacy

Table 1 shows that health literacy was approximately two points higher for parents compared to the children's NVS scores out of a total possible of six points. However, neither parent nor child health literacy scores varied between the two groups at baseline. Veggie self-efficacy was approximately 65 out of a possible 120 for the total participant group at baseline. There was also no significant difference in Veggie Self-Efficacy scores between groups at baseline.

### Macular Pigment Changes (HFP and MPEYE Assessments)

Macular pigment density across the entire macula, assessed utilizing Haidinger's Brushes degree of polarization showed a significant increase for participants taking a daily 5 mg lutein gummy compared to the placebo group, F (1,73.4)=6.86, p=0.011. Pairwise comparison analyses revealed that the group of participants receiving lutein demonstrated a mean marginal estimate increase in MP-eye values of 1.5 while the group receiving the placebo showed a mean marginal estimate increase of only 0.07 resulting in a marginal mean estimate increase in the treatment group of approximately 1.45 MP-eye units (95% confidence interval is 0.346 to 2.554, Cohen's d=1.85). Based on the absolute changes in means from baseline, the participants taking lutein showed a 13.7% improvement in HB measured macular pigment compared to participants taking placebo. On the other hand, macular pigment peak volume at the center of the fovea, measured as MPOD via heterochromatic flicker photometry, did not demonstrate any increase based on lutein supplementation and in fact remained fairly constant throughout the course of the study for both the lutein and placebo groups, F (1,59.07)=0.044, p=0.835, Marginal mean estimates showed a mean change of 0.044 and 0.039 for the lutein and placebo groups respectively, yielding a marginal mean estimate difference of 0.005 between both groups (95% confidence interval of −0.052 to 0.042, Cohen's d=0.15). Based on the absolute changes in means from baseline, the participants taking lutein showed a 0.05% improvement in HFP measured macular pigment compared to participants taking placebo.

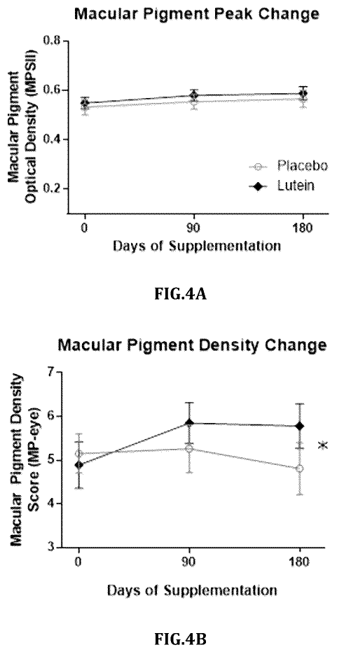

The means±SD of MPOD measurements by HFP are summarized in Table 2 and FIG. 4A. Using HFP, there was no difference between groups at baseline (PL 0.532±0.158 vs. LuT 0.549±0.122), and no significant difference between the interventions at day 90 (PL 0.554±0.156 vs. LuT 0.580±0.116) and day 180 (PL 0.566±0.179 vs. LuT 0.588±0.141). Marginal mean estimates showed a mean change of 0.044 and 0.039 for the lutein and placebo groups respectively, yielding a mean difference of 0.005 between both groups (p=0.835). While MPOD values did increase for both groups over the course of treatment (mean change of LuT 0.039 and PL 0.034), the improvement did not reach statistical significance (p=0.543).

TABLE 2
Macular Pigment Scores Over the Course of the Study Using HFP

| HFP | Placebo | Lutein |
|---|---|---|
| Baseline | 0.532 ± 0.158 | 0.549 ± 0.122 |
| | (n = 26) | (n = 27) |
| Day 90 | 0.554 ± 0.156 | 0.580 ± 0.116 |
| | (n = 27) | (n = 26) |
| Day 180 | 0.566 ± 0.179 | 0.588 ± 0.141 |
| | (n = 26) | (n = 26) |

The means±SD of MPOD measurements by MPEYE are summarized in Table 3 and FIG. 4B. Using MPEYE, there was no significant difference between the interventions at baseline (PL 5.15±2.35 vs. LuT 4.89±2.76, p=0.023). However, statistically significant differences emerged between the interventions at day 90 (PL 5.26±2.84 vs. LuT 5.85±2.45) and day 180 (PL 4.81±3.10 vs. LuT 5.78±2.68).

TABLE 3
Macular Pigment Scores Over the
Course of the Study Using MPEYE

| MPEYE | Placebo | Lutein |
|---|---|---|
| Baseline | 5.15 ± 2.35 | 4.89 ± 2.76 |
| | (n = 27) | (n = 27) |
| Day 90 | 5.26 ± 2.84 | 5.85 ± 2.45 |
| | (n = 27) | (n = 27) |
| Day 180 | 4.81 ± 3.10 | 5.78 ± 2.68 |
| | (n = 27) | (n = 27) |

The MMRM revealed a statistically significant effect for treatment but not for visit or the treatment by visit interaction (Table 4). Further, pairwise comparison analyses revealed that the group of participants receiving lutein demonstrated an average increase in MPEYE values of 1.5 while the group receiving the placebo showed an overall change of 0.07 resulting in an increase in the treatment group of approximately 1.45 units, p=0.011 with a 95% confidence interval of 0.346 to 2.554 based on estimated marginal means and a moderate effect size (Cohen's d=0.31) based on mean group differences at Day 180.

TABLE 4
Mixed Models Repeated Measures Analysis of Macular Pigment

| | | | p-value | 95% |
|---|---|---|---|---|
| Fixed Effect | Fixed Effect | Standard | (based on t- | Confidence |
| Parameter | Estimate | Error | statistic) | Interval |
| Treatment | −.013 | .030 | .663 | −.073 to .047 |
| Visit | −.019 | .026 | .460 | −.071 to .033 |
| Treatment × | .017 | .037 | .655 | −.057 to .090 |
| Visit | | | | |
| Treatment | −1.635 | .707 | .023* | −3.036 to −.234 |
| Visit | .074 | .621 | .905 | −1.173 to 1.321 |
| Treatment × | .370 | .878 | .675 | −1.393 to 2.134 |
| Visit | | | | |

*statistically significant

Participant scores on both of the psychophysical methods for measuring macular pigment (HFP and HB) were correlated at baseline, r (58)=0.335, p=0.010, but were no longer correlated following the six months of supplementation, r (52)=0.180, p=0.201, indicating that the two methods may be measuring different underlying physiological parameters.

### Digital Eye Strain

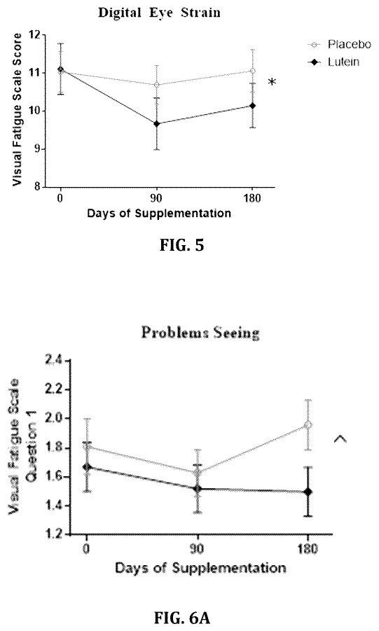

Evaluation of the results revealed that participants supplemented with 5 mg of lutein over the six-month study showed an overall treatment effect demonstrating a statistically significant reduction in digital eye strain compared to the participants taking only a placebo, F (1,107)=6.798, p=0.010. Evaluation of the estimated marginal means revealed a reduction in visual fatigue of 1.184 (SE=0.280) in the group taking lutein and a much smaller decrease in visual fatigue of 0.148 (SE=0.282) in the participants taking the placebo gummy for a mean difference of 1.036 between the two groups (95% confidence interval of 0.249 to 1.823, Cohen's d=1.85). As a reference, a study in healthy adults found a one-to-two-point decrease in visual fatigue following approximately one hour of reading on LCD, e-ink or paper formats using the same VFS measurement tool (Benedetto et al., 2013). For the current study, the participants taking lutein showed a 5.56% improvement in visual fatigue compared to participants taking placebo based on the absolute changes in means from baseline (FIG. 5). There was no significant visit or treatment by visit effect.

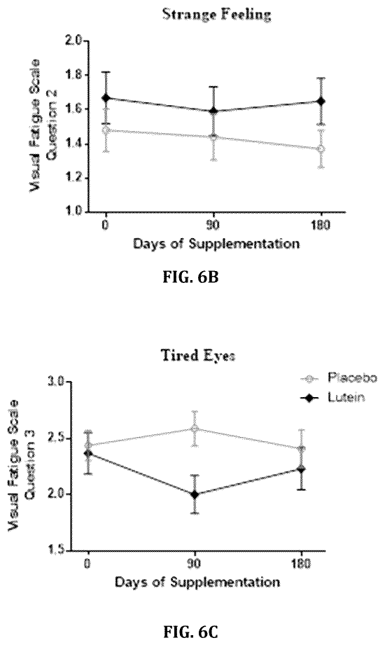

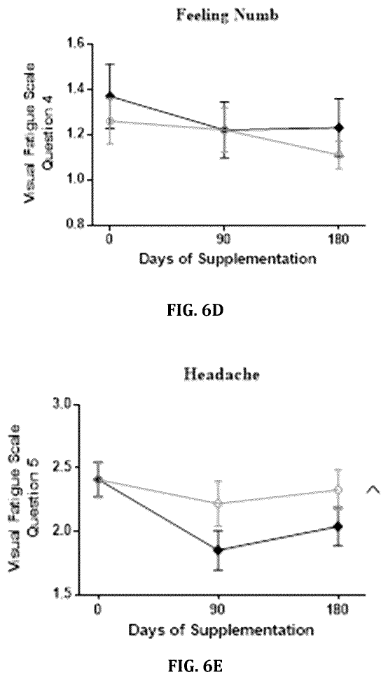

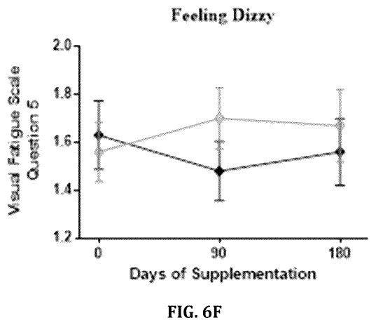

Post hoc independent sample t-tests of each individual question of the VFS, indicates that the responses to “I have problems seeing” and “I have headaches” could potentially be driving the benefits observed with the lutein supplementation (FIGS. 6A-6F).

### Health Awareness

Participants' abilities to obtain and use information to make informed health decisions was significantly correlated with the same capabilities of their parents as seen by the significant correlations of parents' and participants' health literacy scores, r (41)=0.568, p<0.001. Interestingly, parents' health literacy scores, but not the children's scores were significantly correlated with baseline macular pigment values, indicating that parents may still play a pivotal role in adolescent nutrient intake. Of special note, the parents' scores were only significantly related to HB baseline macular pigment values and not to HFP baseline values, again indicating that the HB methodology may be a more sensitive technique for assessing total macular pigment quantities, r (41)=0.350, p=0.025 and r (40)=−0.105, p=0.521, for parent health literacy and baseline macular pigment HB and HFP values respectively.

### DISCUSSION

The present study evaluated the effects of a six-month daily intervention of 5 mg lutein on macular pigment levels and digital eye strain in a healthy adolescent population exposed to four hours or more of daily digital device use. The findings demonstrate that a moderate nutritional intervention of 5 mg lutein (the equivalent of ¼ cup of cooked spinach) was able to increase macular pigment levels in a healthy adolescent population. Following six-months of daily supplementation, participants consuming a 5 mg lutein gummy demonstrated a 14% increase in macular pigment density compared to those taking a placebo gummy. Along with the macular pigment increase, participants in the treatment group reported a 6% decrease in digital eye strain compared to the levels reported by participants in the placebo group. Health literacy was assessed at baseline for both the participant and the parent guardian as one potential indicator of healthy lifestyle habits. Measures of health literacy point to the possible influence of parents' health knowledge and utilization on a child's macular pigment levels even into the teenage years. While the parent and child's health literacy scores were significantly correlated with each other at baseline, it was the parent's health literacy score and not the child's that was significantly correlated with the participant's baseline macular pigment levels while the child's own scores were not. The relationship between parent health knowledge and child macular pigment levels was no longer statistically significant by the end of the nutritional intervention study. Finally, the results indicate that a simple, non-invasive method for assessing macular pigment levels based on the principles of Haidinger's brushes degree of polarization may provide a practical and cost-effective method for tracking macular carotenoid levels in children, pre-teens and teenagers and is sensitive to moderate lifestyle changes such as a small increase in daily carotenoid intake.

To the inventors' knowledge, this is the first study to show that lutein supplementation of 5 mg per day in healthy preteen and teenage children leads to an increase in macular pigment levels and reduced digital eye strain compared to a placebo group. While more than 30 studies have shown increases in macular pigment following lutein supplementation in adults, no intervention trials have been conducted to date in healthy children and adolescents (Hu et al., 2023b; Ponce-García et al., 2024). A meta-analysis of clinical studies on macular pigment and children identified a total of 13 studies in children between the ages of 0 and 12 years old but failed to find any studies conducted in children older than 12 years of age (Ponce-García et al., 2024). Even more importantly, none of the 13 studies reported were interventional trials. While the cross-sectional studies uncovered important relationships between macular pigment levels and visual and cognitive outcomes, an interventional trial is required to build the causal relationship between lutein and health outcomes. To date, only one published study is available reporting lutein supplementation in children (Parekh et al., 2024). The study was conducted with undernourished Indian children between 5 and 12 years of age and investigated the effects of 10 mg lutein and 2 mg zeaxanthin per day for 6 months on visual and cognitive outcomes (Parekh et al., 2024). The findings from the present study differ in that the current study utilized a lower dose of 5 mg lutein and supplemented healthy older children and teenagers between 8 and 16 years of age. This is significant in that it is the first study to investigate this question and be able to show a significant improvement in macular pigment and digital eye strain compared to a placebo group in this age group and with a low dose of lutein supplementation. Additionally, this is the first study to compare the two psychophysical measurement techniques of HB and HFP, the first to find differences in outcomes between the two groups and the first to use the MPeye device in a human interventional study with a placebo group. To date, the MPeye device, which relies on the HB technique for assessing macular pigment in the retina, has only been utilized in cross-sectional, epidemiological single arm studies (Sangani et al., 2024; Temple et al., 2019a).

More than 30 clinical studies have shown macular pigment increases with lutein supplementation in adults (Hu et al., 2023b). However, not all studies in adults have been able to demonstrate macular pigment improvements even at doses as high as 20 mg per day (Yoshida et al., 2023). In the present study, participants exhibited a 14% increase in macular pigment at a daily dosage level of 5 mg lutein compared to participants taking only placebo. There are several potential explanations for this finding. It is possible that the delivery format of a daily gummy provides a more bioavailable system for the fat-soluble lutein to be digested and absorbed by the body, thus resulting in the significant outcomes observed (Bhat & Mamatha, 2021; Böhm et al., 2021; Olmedilla-Alonso et al., 2021; Thürmann et al., 2005). In addition, the specific source of the lutein could be a higher-quality ingredient compared to lutein sources used in other human clinicals that failed to report significant improvements (Chung et al., 2004; Dai et al., 2021; Lim et al., 2021; Sheshappa et al., 2015). In addition, it is possible that the preteen and teenage eye is in a state of rapid growth and development and may be better able to absorb all sources of supplemental lutein more readily than the fully formed adult retina (Cheng & Tian, 2024; Ho et al., 2024). For example, it is possible that the preteen and teenage eye have higher amounts of the lutein transporter protein, StAR-related lipid-transfer protein 3 (StARD3), and therefore are able to absorb higher amounts of lutein circulating in the serum (Arunkumar et al., 2020b; Horvath et al., 2016; Sluchanko et al., 2022; Tanprasertsuk et al., 2016). Whatever the underlying mechanism, the data from the present study indicate that supplementing healthy preteen and teenage children with at least 5 mg lutein a day may be an effective way to increase macular pigment levels in children's eyes. Furthermore, given that the macular pigment increases were seen across the breadth of the macula as measured with the HB device and not necessarily just in the center peak as assessed by the HFP method (Forster, 1954; Manion & Stokkermans, 2024; Mottes et al., 2022; Temple et al., 2019a), the results of the study point to the possibility that lutein may accumulate differently in the retina of children compared to macular accumulation in the adult eye. Additionally, as the first study to use the HB technique via the MPeye device to assess macular pigment levels following lutein supplementation, the current findings could mean that the MPeye device is more sensitive to changes in overall macular pigment levels and may serve as an ideal device for rapid screening of lutein levels in both children and adults.

A secondary research question in the current study was whether lutein supplementation could lead to a reduction in digital eye strain in healthy teen and preteen children exposed to digital devices for four hours or more per day. Digital eye strain was assessed via the Visual Fatigue Scale (VFS), a validated tool that measures digital stress on a scale from 6 to 24. In the present study, participants taking 5 mg of lutein daily demonstrated a one-point improvement on the VFS scale, corresponding to a reduction in digital eye strain of 5.56% compared to the change seen in the placebo group. For comparison, a study in healthy adults found a one-to-two-point worsening of visual fatigue following approximately 1 hour of reading on LCD, e-ink or paper formats using the same VFS measurement tool (Benedetto et al., 2013). Researchers have suggested the 20/20/20 rule as an intervention to reduce digital eye strain in which a person takes a break every 20 minutes and spends at least 20 seconds looking at something 20 feet away (Bin Maneea et al., 2024; Datta et al., 2023). Multiple research studies have been conducted to measure visual fatigue using a similar survey tool known as the Computer Vision Syndrome questionnaire (CVS-Q; Seguí Mdel et al., 2015). One interventional study of the 20/20/20 rule found a 2-point improvement in the CVS-Q score for individuals following the 20/20/20 rule for a 2-week duration indicating that the 1-point difference observed in the present study may be clinically meaningful in an individual's daily life experience (Seguí Mdel et al., 2015; Talens-Estarelles et al., 2023). However, other research investigations have failed to demonstrate significant improvements in digital eye strain based on various intervention techniques. For example, a study using blue blocking lenses failed to find reductions in subjective symptom scores for visual fatigue among healthy adults (Palavets & Rosenfield, 2019; Singh et al., 2021). Visual fatigue and digital eye strain have been recognized as a serious health problem for over 20 years (Rosenfield, 2016; Sheppard & Wolffsohn, 2018) with increased concerns following the rise in digital device exposure in more recent years (Jaadane et al., 2015b; Mylona et al., 2023). For instance, a study of 10,000 medical students conducted in 2024 revealed a computer vision syndrome prevalence rate of approximately 57% (Iqbal et al., 2024).

One key finding of the study was that parents' health literacy scores were significantly and positively correlated with their children's health literacy scores as well as to the child's macular pigment levels at baseline. It's important to note that the children's own health literacy scores were not correlated to their macular pigment levels, indicating that parents of children between 8 and 16 years of age may play a more significant role in healthy eating habits of kids than the children themselves. This finding implies that efforts to get kids to eat more vegetables may be more effective if directed at the parents and not necessarily to the children. This also indicates the importance of the family as a social factor for the overall health and wellbeing of the child. Additionally, only macular pigment scores using the HB methodologically were significantly correlated to the parent's health literacy values, indicating that the HB method may be a more sensitive measure of macular pigment in the preteen and teenage eye and perhaps even in the eye across the lifespan.

### CONCLUSION

The purpose of this study was to determine the effects of daily lutein supplementation on eye and brain outcomes in healthy preteen and teenage children exposed to daily digital devices. In addition, baseline data were collected to better understand the potential roles of social, biological, and psychological factors on healthy eating behaviors, specifically including lutein-rich fruits and vegetables in the daily diet. Understanding the benefits of supplemental lutein intake in a gummy format is important since the preteen and teenage age group in the United States represents the lowest intake of daily fruits and vegetables and has the lowest healthy eating index scores of all age groups (Dietary Guidelines for Americans, 2020-2025., December 2020.). Lutein cannot be produced by the body and must be obtained from the daily diet or supplementation. The benefits of lutein in adults, pregnant moms, and infants have been studied extensively and shown to be important for both visual and cognitive function. However, to date, no lutein intervention studies have been conducted in healthy preteen and teenage children. Given the rapid developmental changes occurring during the adolescent years, it is essential to understand the role of lutein in both eye and brain development in preteen and teenage children.

There are three key findings from the current study. First, it was demonstrated that 6-months of supplementation with 5 mg lutein in a gummy format can increase macular pigment and thus retinal lutein levels in healthy preteen and teenage children. Second, it was also shown that 6-months lutein supplementation at a 5 mg dose can reduce digital eye strain in healthy teenagers and preteens that are exposed to digital devices for at least 4 hours per day. Finally, the results of the current study revealed that children's health literacy is related to their own parent or guardian's health literacy. Interestingly, parents' health literacy but not the children's health literacy, was correlated with lutein levels in the eyes of the children, indicating an important role of the parent in children's health, even during the preteen and teenage years.

This was the first study to date to provide supplemental lutein to healthy teenage and preteen children and assess outcome changes in eye and brain health. The results confirm that even a daily dose as low as 5 mg can have a positive impact on a teenager's macular pigment levels as well as meaningful reductions in digital eye strain. This finding is important for parents, schools and government organizations setting dietary guidelines for children. While 5 mg of lutein can be obtained from the diet, most children are not eating the necessary fruits and vegetables to reach doses of even 1-2 mg lutein daily. To maximize eye and brain development during the formative adolescent years, it may be helpful to consider ways to increase fruit and vegetable intake in this age group. One method may be to increase healthy literacy rates for parents, as the results of this study indicate that children of parents with high health literacy also had higher lutein levels in their eyes.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

It should be further appreciated that minor dosage and formulation modifications of the composition and the ranges expressed herein may be made and still come within the scope and spirit of the present invention.

The foregoing descriptions have been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplish at least all of the intended objectives.

1. A method of minimizing damage to a foveal architecture of an individual, comprising:
administration of at least one ocular nutrient selected from the group consisting of antioxidant vitamins, carotenoids, antioxidant minerals, and natural antioxidant extracts to the individual, wherein the individual is at risk of damage to the foveal architecture due to exposure to digital screens, blue light, or near work.

2. The method of claim 1, wherein the at least one ocular nutrient is a carotenoid.

3. The method of claim 2, wherein the carotenoid is lutein and/or zeaxanthin.

4. The method of claim 2, wherein the foveal architecture has not been previously damaged.

5. The method of claim 2, wherein the individual has a condition from the group consisting of: damage to the retinal nerve layer, improper abnormal eyeball shape, irregular anatomical development of the fovea, and damage to the foveal architecture.

6. The method of claim 2, wherein the individual is a child, preteen, teen, or young adult.

7. A method of minimizing abnormal eyeball shape change in an individual, comprising:
administration of at least one ocular nutrient selected from the group consisting of antioxidant vitamins, carotenoids, antioxidant minerals, and natural antioxidant extracts to the individual, wherein the individual is at risk of abnormal eyeball shape due to exposure to digital screens, blue light, or near work.

8. The method of claim 7, wherein the at least one ocular nutrient is a carotenoid.

9. The method of claim 8, wherein the carotenoid is lutein.

10. The method of claim 8, wherein the individual has a normal eyeball shape.

11. The method of claim 8, wherein individual has a condition from the group consisting of: damage to the retinal nerve layer, improper eyeball elongation, abnormal eyeball shape, irregular anatomical development of the fovea, and damage to the foveal architecture.

12. The method of claim 8, wherein the individual is a child, preteen, teen, or young adult.

13. A method of reducing eye conditions in an individual, comprising:
administration of at least one carotenoid, wherein the individual is at risk for developing at least one eye condition due to exposure to digital screens, blue light, or near work.

14. The method of claim 13, wherein the at least one eye condition is selected from the group consisting of: vision, eye strain, accommodative fatigue, dry eye, strabismus, damage to foveal architecture, irregular development of fovea, eyeball shape change, improper eyeball elongation, screentime fatigue, biomechanical stress, and retinal nerve cell layer damage.

15. The method of claim 14, wherein the individual is already suffering from at least one eye condition, wherein administration of the at least one carotenoid reduces an effect of the at least one eye condition.

16. The method of claim 14, wherein the individual is not yet suffering from at least one eye condition, wherein administration of the at least one carotenoid prevents or delays the at least one eye condition.

17. The method of claim 14, wherein the at least one carotenoid is administered at a level sufficient to increase macular pigment levels.

18. The method of claim 16, wherein the carotenoid is administered between 0.0001 and 2 mg/kg body weight.

19. The method of claim 14, wherein the carotenoid is lutein and/or zeaxanthin.

20. The method of claim 14, wherein the individual is a child, preteen, teen, or young adult.
